(19) United States
(12) Patent Application Publication
(10) Pub. No.: US 2003/0194709 A1
(43) Pub. Date: Oct. 16, 2003

(54) Hydrophobic zone device

(76) Inventor: Xing Yang, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/121,214

(22) Filed: Apr. 10, 2002

(51) Int. Cl. C12Q 1/68; G01N 33/543; H01L 21/00

(52) U.S. Cl. 435/6; 436/518; 438/1

(57) ABSTRACT

A method is provided for making and using an assay chip having a hydrophilic region bounded by a hydrophobic region. This is desirable because it allows the user to deposit reagents in an aqueous medium on the hydrophilic region while the hydrophobic region prevents the reagents from flowing away from the hydrophilic region. Hence, the reagents can be isolated in the hydrophilic region to minimize any loss or dilution of the reagents. In a preferred embodiment, the chip surface features a plurality of hydrophilic regions bounded by hydrophobic regions allowing the user to conduct a plurality of assays on the same chip without cross-contamination of the samples. This device is of particular interest to the field of genetic analysis in which oligonucleotides are attached to a gold electrode for electrochemical analysis.

### BACKGROUND OF THE INVENTION

1. Field of the Invention

This invention relates to genetic analysis chip having a hydrophobic zone, preferably bounding a hydrophilic zone in which a genetic sample can be analyzed.

2. Description of the Related Art

In the field of genetic analysis, there are several kinds of DNA chips. Although they are all referred to as “DNA chips,” they can be quite different from each other.

One kind of DNA chip is a DNA microarray or GENECHIP™ (a trademark of Affymetrix). These chips are typically a synthetic polynucleotide array on a substrate. The substrate could be glass, silicon (covered with silicon dioxide), polymer, etc. The polynucleotide array is synthesized on the substrate using technologies based on photolithography (Affymetrix, U.S. Pat. No. 5,143,854, U.S. Pat. No. 5,405,783 U.S. Pat. No. 5,445,934), inkjet printing (Agilent Technologies), electrochemistry (CombiMatrix, U.S. Pat. No. 6,093,302), or maskless light-directed fabrication (NimbleGen). See S. Singh-Gasson, R. Green, Y. Yue, C. Nelson, F. Blattner, M. Sussman, and F. Cerrina, “Maskless Fabrication of Light-Directed Oligonucleotide Microarrays Using a Digital Micromirror Array,” Nature Biotechnology, Vol. 17, pp. 974-978, October, 1999, all of which are hereby expressly incorporated by reference. The analysis is usually based on hybridization. The analyte nucleic acid, or “target” is incubated with the DNA array, and the extent of hybridization with each DNA probe on the array is assessed in order to identify those which are perfect complements to the target. This requires the preparation of a fragmented and labeled target mixture from a genetic sample. Confocal epifluorescence scanning is used in conjunction with fluorescent labeling to monitor hybridization. The sample preparation step, which involves processing of various reagents, is performed either manually off the chip or in an integrated polycarbonate cartridge. See R. C. Anderson, X. Su, G. J. Bogdan, and J. Fenton, “A Miniature Integrated Device for Automated Multistep Genetic Assays,” Nucleic Acids Research, Vol. 28. No. 12, 2000.

In U.S. Pat. No. 6,221,586 (hereby expressly incorporated by reference), Barton describes compositions and methods for electrochemical detection of base stacking perturbations within oligonucleotides duplexes adsorbed onto electrodes. Specifically, that technology utilizes an intercalative, redox-active moiety attached to a DNA duplex immobilized on an electrode. Electrical current is then made to flow along the duplex. Interruptions caused by base-stacking perturbations are detectable based on measurements of the electrical resistance of the duplex.

In the manufacture of many of these types of DNA chips, a liquid containing reagent DNA is deposited on a substrate, and the liquid is removed (as by evaporation), leaving the reagent DNA on the chip in a discrete, defined area. Maintaining the liquid in the desired defined zone can be problematic. One attempt to provide DNA arrays formed by depositing droplets of aqueous liquid is disclosed in U.S. Pat. No. 6,210,894 to Brennan. This patent discloses arrays of functionalized binding sites on a substrate, with derivatized hydrophilic binding sites surrounded by hydrophobic regions.

A significant issue in chips having hydrophobic zones or regions is wettability of the chip during performance of the assay. It is often desirable to flood the entire surface of the chip with a common solution, such as a sample solution, wash solution, buffer, or reagent solution. Hydrophobic surfaces can understandably interfere with such assay steps. Moreover, masking and etching steps for depositing or removing hydrophobic layers are not always desirable. Finally, many assays require that the reagents on the chip are attached, directly or indirectly, to electrodes. At least some of these issues are addressed by the present invention.

### SUMMARY OF THE INVENTION

One aspect of the invention is a hydrophobic zone on a genetic analysis chip. Preferably the hydrophobic zone bounds a hydrophilic zone in which a reagent sample can be analyzed.

Another aspect of the invention is a method for positioning a plurality of droplets on electrodes, including providing a substrate having a plurality of electrodes onto which droplets can be positioned in a plurality of hydrophilic zones, wherein each hydrophilic zone is bounded by a hydrophobic zone; and applying discrete aqueous droplets into a plurality of the hydrophilic zones. Preferably, the hydrophobic zone contains a fluoropolymer. The hydrophobic zone can be a line that is continuous and completely encircles the hydrophilic zone. Alternatively, the hydrophobic zone can be a broken line. In either case, the hydrophilic surface of the substrate can be exposed both inside of and outside of the hydrophobic line. Preferably, the hydrophobic zone is defined by depositing a hydrophobic material on the surface of the chip and then etching away a portion of it.

In another aspect of the invention, the deposited droplets described above contain reagents, and can be applied to different zones on the substrate for the performance of an assay. The reagents can contain DNA, RNA, an enzyme, an antigen, a peptide, a peptidomimetic, an antibody, other types of specific binding molecules, a substrate, a native, recombinant, or chimeric receptor, a chemical reagent, a redox moiety, a chemical or biological sensor or sensor molecule, an organic chemical compound, and the like. In a preferred embodiment, the reagents contain DNA. In a further aspect of the invention, the reagents can be dried on the substrate such that different dried reagents are provided in different hydrophilic zones.

Another aspect of the invention is an assay surface, including: a plurality of spatially discrete reagent zones, each comprising at least one reagent, wherein the reagent zones are relatively hydrophilic; and a relatively hydrophobic line surrounding each of the reagent zones. This assay surface can further include relatively hydrophilic regions located outside of the hydrophobic lines, which do not contain a reagent. Preferably, assay reagents are deposited on the assay surface. In a preferred embodiment, the assay reagents contain DNA. Different reagents can be located in different reagent zones. In creating the assay surface, the substrate can contain a silicon wafer. Further, the assay surface can contain a plurality of electrical conductors in physical and/or electrical contact with the reagent zones. Preferably, each reagent zone is in contact with a different electrical conductor. Additionally, a continuous liquid layer can overlay a plurality of the reagent zones. Further, an external electrode can be placed in contact with the liquid layer thus completing a circuit and allowing an electrochemical measurement to be made on the reagents.

Another aspect of the invention is a method for performing an assay, including: providing an assay surface featuring a plurality of reagent zones, each reagent zone surrounded by a hydrophobic material, wherein a reagent is bound to the assay surface at the reagent zone, and hydrophilic areas are located on the surface both inside of and outside of the hydrophobic material; flooding the assay surface with a liquid sample, such that a layer of liquid covers the assay surface; and detecting an interaction between an analyte, if present, and the reagent in a reagent zone. Preferably, the interaction of the reagent and the analyte produces an electrical signal measurable in said reagent zone. Preferably, the electrical signal is measured through one or more of a plurality of first electrodes in electrical contact with the reagent zones and one or more second electrodes in electrical contact with the liquid sample. The second electrodes can be located remotely from the reagent zone in which the electrical signal is produced.

Another aspect of the invention is an assay device, including: a substrate having a surface including a plurality of reagent-bearing zones, wherein the reagent-bearing zones are relatively hydrophilic and are each bounded by a relatively less hydrophilic zone, wherein the hydrophilic zones are differentiated from the less hydrophilic zones as a result of the texture of the surfaces. Preferably, the hydrophilic zone is smoother than the less hydrophilic zone. The less hydrophilic zone can also contain a fluoropolymer to enhance its hydrophobicity.

### BRIEF DESCRIPTION OF THE DRAWINGS

FIG. 1 is a cross-sectional view of a DNA chip of the present invention, showing the retention of a liquid droplet within a hydrophobic zone.

FIG. 2 is a top plan view of a DNA chip having electrical contacts within a hydrophilic zone, bounded by a hydrophobic zone.

FIGS. 3a-3h are cross-sections of silicon wafers being manufactured into DNA chips according to the present invention, illustrating the progressive etching and deposition steps in the manufacturing process.

FIG. 4 is a top plan view of a DNA chip of the present invention illustrating possible electrode patterns and hydrophobic layer placement.

FIG. 5 is a top plan view of a DNA chip of the present invention, illustrating an alternative hydrophobic zone arrangement.

FIG. 6 is a top plan view of a DNA chip of the present invention, illustrating another alternative hydrophobic zone arrangement.

FIG. 7 is a cross-section of a DNA chip of the present invention in which the hydrophobic zone is created by microroughening on the surface of the chip.

FIG. 8 is a cross-section of a DNA chip of the present invention in which the hydrophobic zone is created using a both a hydrophobic material and microroughening.

### DETAILED DESCRIPTION OF THE PREFERRED EMBODIMENT

In the present disclosure, various methods and apparatus are provided for preparing assay chips having reagent bound in discrete zones. Although the present disclosure describes the inventions primarily in the context of DNA chips, it will be understood and appreciated that many aspects of the disclosure are applicable to assay chips having various other reagents bound thereto. Thus, in addition to DNA, the bound assay reagent can include, without limitation, an enzyme, RNA, an antigen, a peptide, a peptidomimetic, an antibody, other types of specific binding molecules, a substrate, a receptor, a chemical reagent, a redox moiety, a chemical or biological sensor or sensor molecule, an organic chemical compound, and the like. Thus, except as specifically required in the claims, the references to DNA and DNA chips are to be considered exemplary, not limiting.

In one aspect of the present invention, the assay chip is particularly suited for use in electrochemical analysis. In these embodiments, the invention includes an assay device having a substrate, a relatively hydrophobic zone surrounding a relatively hydrophilic zone, and one or more electrodes located within the hydrophobic zone, with a reagent attached to the one or more electrodes.

A. Chip Design and Fabrication

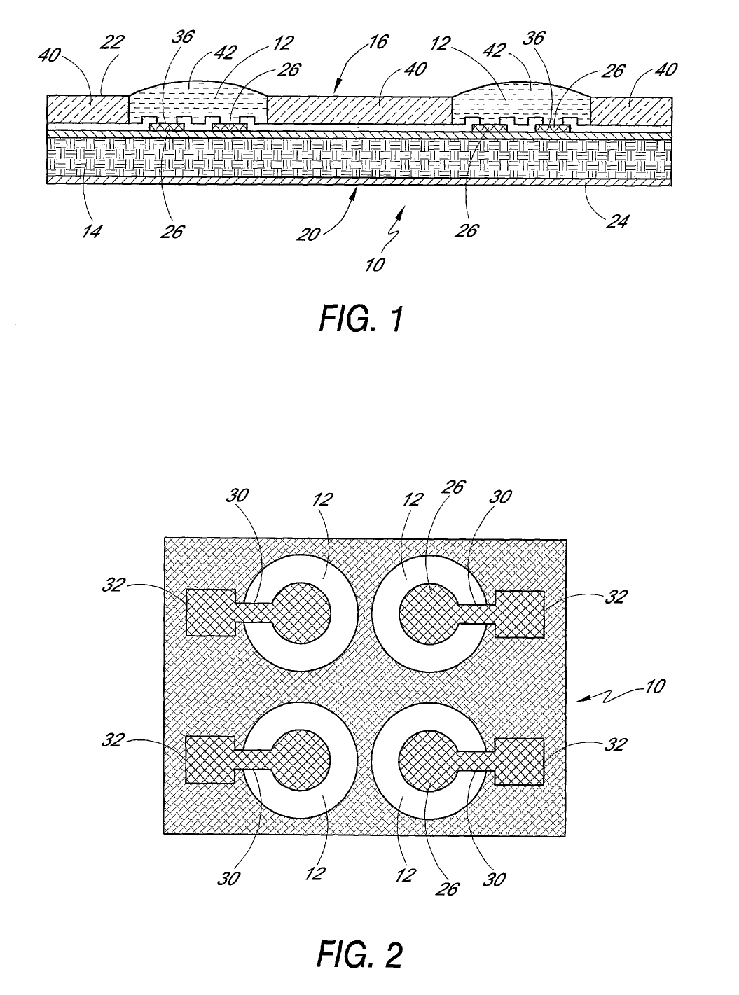

One embodiment of the chip 10 of the present invention is illustrated in FIG. 1. This Figure is a cross-section of a chip 10 having two assay regions 12 on the surface thereof. The illustrated embodiment shows only two regions for ease of illustration, not by way of limitation. It will be understood that in many embodiments of the invention, the chip 10 will have many more assay regions, e.g., 5, 10, 20, 30, 50, 100, 200, 1000 or more regions. These assay regions are preferably arranged into a regular two-dimensional array.

The chip 10 includes a substrate 14 serving as the body of the chip. The substrate can be made of silicon, including monocrystalline and polycrystalline silicon, preferably of semiconductor grade. Alternatively, it can constitute plastic or other polymer material, glass, or composite material, including any of the common printed circuit board materials. In the illustrated embodiment, the substrate 14 preferably includes one or more insulating layers of silicon dioxide or other suitable dielectric material. This is particularly useful when the substrate 14 is silicon, and is not necessarily required when the substrate 14 is itself a dielectric material. In FIG. 1, a substrate 14 is shown, having a top 16 and a bottom 20. A first top insulating layer 22 and a bottom insulating layer 24 are respectively shown on the top 16 and bottom 20 of the substrate. One or more electrodes 26 are formed on top of the first top insulating layer 22. Typically, at least one, and sometimes two or more electrodes 26 are formed in each assay region 12. The first top insulating layer 24 insulates the electrodes from the silicon substrate. The electrodes are advantageously formed of gold or other noble metal, but may be any conductive material onto which reagent may be affixed, including without limitation, platinum, palladium, rhodium, carbon electrodes such as glassy carbon, oxide electrodes, or semiconductor electrodes. The electrodes may also contain conductive polymers on the surface. Gold electrodes are particularly preferred. The electrodes 26 are joined to electrical conductors 30 that form a conductive path to a desired connection point or electrical contact 32 (see FIG. 2).

Preferably, a second top insulating layer 34 is formed over the first top insulating layer 22 and the electrical conductors 34, isolating the electrical conductors 30 from exposure on the surface of the chip 10 during performance of the assay. The second top insulating layer 34 may advantageously be formed of silicon dioxide, but other insulating materials, including polymers, may be used in various embodiments of the chip 10. For example, if the substrate 14 is a printed circuit board substrate, a conformal insulating coating may be used. Windows 36 are preferably patterned in the second top insulating layer 34 to provide fluidic and electrical connections to the electrodes 26.

A hydrophobic layer 40 is advantageously provided on top of the chip 10 and over the second top insulating layer 34. This hydrophobic layer 40 is one manner in which the present invention provides droplet control on the surface of the chip 10. During fabrication of the chip, a plurality of different reagents may advantageously be deposited into the different assay regions 12 of the chip 10. These reagents are typically contained in microdroplets 42 of a liquid, preferably an aqueous liquid, and thus dry very quickly to deposit the reagent onto the surface of the assay regions 12 and the electrodes 26. However, despite their small size and rapid drying, they can still spread onto undesired regions of the chip 10 unless some form of droplet control is operational. The hydrophobic layer 40 serves to constrain the droplets 42. The hydrophobic layer 40 illustrated in FIG. 1 surrounds the assay region and provides such a method of droplet control, preventing spreading or diffusion into other assay regions or commingling of different droplets 42. By surrounding the assay region 12 with a hydrophobic layer 40, the chip surface exhibits different wettability based on the hydrophobicity difference between the hydrophobic layer and silicon dioxide or gold.

The hydrophobic layer 40 may advantageously be formed of any material that is more hydrophobic or less hydrophilic than the surface inside the assay region 12. Some suitable materials include fluorocarbons, such as fluorocarbon polymers. Such polymers are well-known to exhibit exceptional hydrophobicity. Alternatively, other hydrophobic materials may also be used, including various organic polymers. One particularly suitable fluoropolymer that can be used in the present invention is a cyclized transparent optical polymer obtained by copolymerization of perfluoro (alkenyl vinyl ethers), sold by Asahi Glass Company under the trademark CYTOP. This material has hydrophobic properties very similar to those of polytetrafluoroethylene, but is soluble in certain perfluorinated solvents and can be applied in thin layers to a substrate. CYTOP is available in the United States through Bellex International Corporation, Wilmington, Del. The CYTOP material designated CTL-809M is particularly preferred for spin-coating applications.

In one preferred embodiment, the hydrophobic layer 40 is applied in a continuous layer over the entire surface (or at least a defined region) of the chip 10, and is then removed in selected locations. Specifically, the hydrophobic layer 40 is advantageously removed to expose the assay regions 12 and the electrodes 26. In comparison to the hydrophobic layer, the electrodes and the silicon dioxide in the assay regions 12 can be easily wetted by the aqueous reagents while the area covered with the hydrophobic layer 40 cannot. This controlled surface property helps to put down different DNA molecules or other reagents with different sequences into different assay regions 12 (and onto different electrodes 26) on the chip.

FIG. 2 illustrates a simple version of a chip 10 of the present invention having four assay regions 12. As mentioned above, most designs of the chip 10 will have many more assay regions. In the illustrated embodiment, the electrodes 26 are joined to electrical contacts 32 by relatively short conductors 32; however, this is simply for purposes of illustration. In practice, the conductors 32 may be much longer, and may traverse the thickness of the substrate 14 or extend to an edge or (in the form of wires) to separate instrumentation or circuitry.

By using a precisely controlled robotic system, drops of solution with DNA molecules in precise volume can be deposited onto some or all of the assay regions. Robotic or computer-controlled spotting devices can be used for this process. Because the openings are isolated from each other, DNA molecules with different sequences (or other different reagents) can be deposited onto adjacent assay regions without mixing.

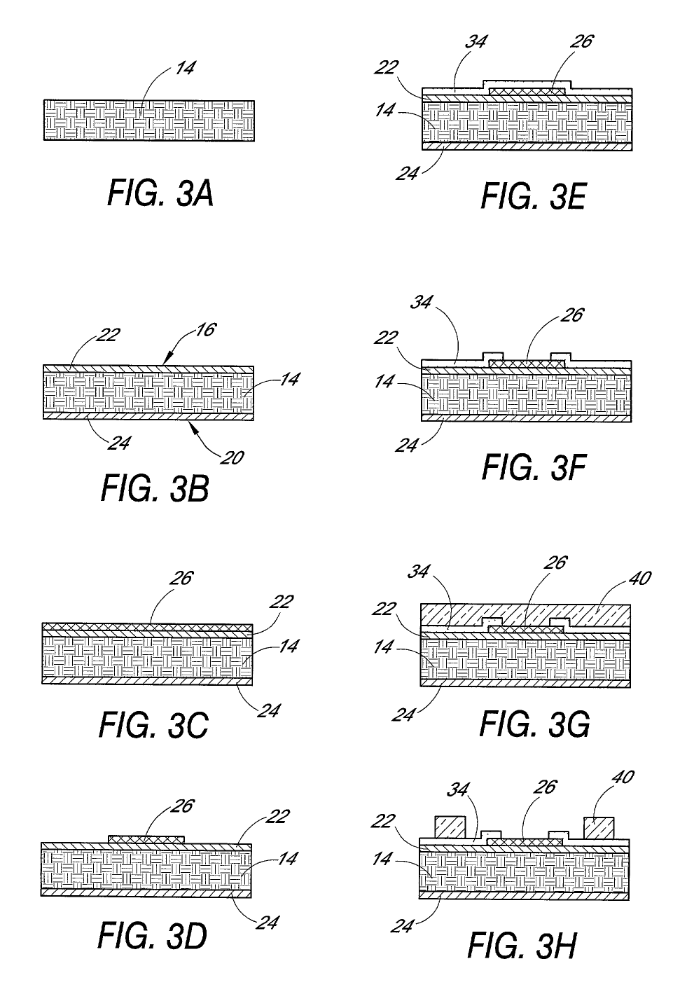

FIGS. 3A-3H illustrate the progressive stages of one exemplary fabrication process using silicon wafers. The process starts with 4 inch single crystalline silicon wafer substrate 14 with <100> orientation. First, with reference to FIG. 3B, top and bottom layers 22 and 24 of 1.5 μm thick silicon dioxide are grown on the top 16 and bottom 20 of the wafers at 1050° C. for 6 hours. Next, with reference to FIG. 3C, a layer 26 of 100 Å chromium and 3000 Å gold is thermally evaporated onto the wafers 14. The chromium layer serves as the adhesion layer to improve the adhesion of gold to silicon dioxide.

Next, with reference to FIG. 3D, the chrome/gold layer is then patterned and etched with chrome and gold etchants to define the electrodes 26 and conductors 30 (as well as, optionally, electrical contacts 32). After that, as illustrated in FIG. 3E, a layer of 3000 Å thick silicon dioxide is deposited on the wafers in a low pressure chemical vapor deposition (LPCVD) reactor at 450° C. for 30 minutes, to form a second top insulating layer 34. This layer of silicon dioxide is often referred as low temperature oxide (LTO) in the semiconductor industry. The LTO layer 34 is then patterned and etched with buffered hydrofluoric acid to expose the gold electrodes, as shown in FIG. 3F.

With reference to FIG. 3G, a layer of 1 μm thick CYTOP, an amorphous fluorocarbon polymer from Asahi Glass Company (with hydrophobic properties similar to polytetrafluoroethylene), is then spin coated on the wafer and cured at 180° C. for one hour, forming the hydrophobic layer 40. The CYTOP layer 40 is patterned and etched with oxygen plasma to define the windows 36 and thus the assay region 12. Preferably, the CYTOP layer is etched such that a ring of CYTOP is left surrounding an electrode 26. This ring thereby divides two hydrophilic zones, one inside the ring and one outside. More preferably, at least one ring surrounds each of a plurality of electrodes thereby creating a boundary around each electrode in which an aqueous sample can be held and isolated from other similarly bounded aqueous samples. Finally, the wafers are diced and ready for testing.

The CYTOP or other hydrophobic layer 40 on the chip 10 serves the function of surface tension control. Experimental study shows that individual buffer solution drops can be easily formed inside the Teflon openings, as shown in FIG. 1. This allows the user to deposit different DNA molecules or other reagents on different electrodes.

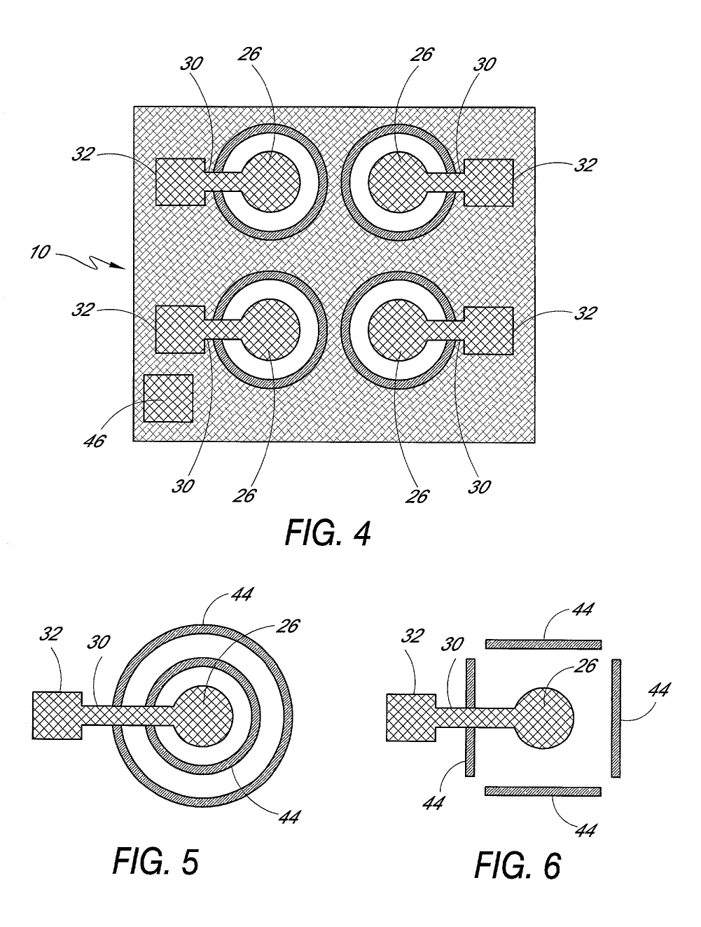

One aspect of the present invention is the ability to wet the entire top surface 16 of the chip 10 during the performance of the assay, or at least the entire portion thereof in which assay regions 12 or electrodes 26 supporting reagent are located. Because some assays further require that after the DNA molecules are deposited, buffer solution, genomic sample, and other reagents have to reach all the electrodes on the chip, the hydrophobic ring is preferred. This embodiment is shown in plan view in FIG. 4. In this embodiment, one hydrophobic ring is made around each electrode 26. Alternatively, as shown in FIG. 5, multiple rings around a single electrode could also be used to further assure containment of an aqueous sample. Finally, as shown in FIG. 6, the hydrophobic layer 40 making up the hydrophobic ring need not necessarily be continuous, but can instead form a discontinuous shape, so long as sufficient hydrophobic material 40 surrounds the electrode 26 to provide droplet control.

With the use of a ring or line of hydrophobic material surrounding the assay region 12 in which the electrode 26 is located, when a droplet of reagent is deposited on top of the electrode, the ring 44 will keep the reagent droplet inside as long as the volume of the droplet is sufficiently small. However, such droplet control is often desired only during manufacture of the chip. During the performance of the assay, it may be desirable to flood all of the surface of the chip, or at least a plurality of assay regions 12, with a single reagent, liquid, or sample, which is preferably continuous and uniform. Because of the relatively small surface area of a ring, much of the chip surface is hydrophilic, the reagents can be easily distributed to the whole chip surface. Note that this is in contrast to the result when the entire assay surface (except for discrete assay regions) is coated with a hydrophobic layer, as in U.S. Pat. No. 6,210,894. That arrangement provides significant difficulties in wetting the entire chip surface, or in bringing a single liquid into contact with all the assay regions.

Note that in the performance of an assay of the type described in U.S. Pat. Nos. 6,221,586 or 5,591,578 (both of which are expressly incorporated herein by reference), it is desirable to flood a plurality of assay regions 12, each with one or more electrodes 26 therein, with a common liquid. As illustrated in FIG. 4, the surface of the chip 10 may advantageously include one or more common electrodes. (The term “common” does not infer any particular polarity, which may vary depending on assay type, but rather denotes that this common electrode 46 completes a circuit with more than one of the electrodes 26 in the assay regions 12, and preferably with all of the various electrodes 26 in the various assay regions 12. Thus, the assay device of the present invention can produce an electrical signal in an assay region 12, which flows through the electrode 26 in that region, wherein an electrical circuit is completed between the common electrode 46 and one or more assay electrodes 26 through an aqueous liquid flooding the surface of the chip 10 during the performance of the assay. So long as this aqueous liquid is making contact with a plurality of said electrodes 26 and/or 46, it is considered a “layer,” regardless of its thickness. Moreover, it is not essential that the layer be an aqueous layer; indeed, any conductive liquid, fluid, or layer providing the necessary conductivity for any particular assay is contemplated in the present invention.

Typically, in the performance of the assay, an interaction occurs between an analyte and a reagent in the assay region 12, which can also be considered a reagent zone or a hydrophilic zone. In many suitable assays, this interaction creates or causes an electrical signal, such as an electrical current. See, e.g., U.S. Pat. Nos. 6,221,586 and 5,591,578. Moreover, in these and other assays, the reagent is attached through covalent or noncovalent means in the assay region 12, preferably to the electrode 26. While many techniques are known for effecting such attachment (e.g., antibody, avidin/biotin, or other specific interactions, hydrostatic interactions, hydrogen bonding, various covalent attachment schemes), one particularly preferred method for attachment when using a gold electrode is the gold/thiol interaction. As more specifically described in the above references, polynucleotide derivatized with a thiol group readily reacts with and attaches to gold surfaces. In one preferred embodiment, one strand each of a plurality of double-stranded DNAs are attached to a gold electrode using such thiol-mediated attachment. This results in a unique, tightly packed, ordered DNA monolayer. Then, as more fully set forth in U.S. Pat. No. 6,221,586, the non-thiol-derivatized strand of each duplex is removed, leaving an ordered array of single stranded DNA capture reagents on the gold electrode. This ordered molecular array is sufficiently cohesive and/or continuous as to substantially prevent contact between the gold electrode and moieties in solution having a charge opposite to that of DNA.

In the fabrication process described above, many other alternative materials and processes can be used. First, the substrate can be glass or other ceramic material, which preferably is flat and smooth. Second, the bottom thermally grown silicon dioxide can be replaced by silicon nitride, silicon dioxide deposited by other means, or other polymer materials provided that they are sufficiently smooth and can stand the high temperature in the following evaporation step. Third, the conducting layer need not be gold, but can be any appropriate material such as platinum, palladium, rhodium, a carbon composition, an oxide, or a semiconductor. If gold is chosen, the layer can be evaporated, sputtered, or electroplated, provided that it is sufficiently smooth to allow DNA molecules or other reagents to be deposited on it. Fourth, the LTO layer can be replaced by spin-on dielectric materials (commonly used in semiconductor industry) or other polymer materials such as polyimide, Parylene, and etc. Fifth, other materials such as Teflon AF amorphous fluoropolymer from DuPont or modified Parylene can be used as the hydrophobic layer. Finally, the temperatures, times, and dimensions specifically recited herein can be altered to produce chips having substantially the same properties and functionality as will be appreciated by those of skill in the art.

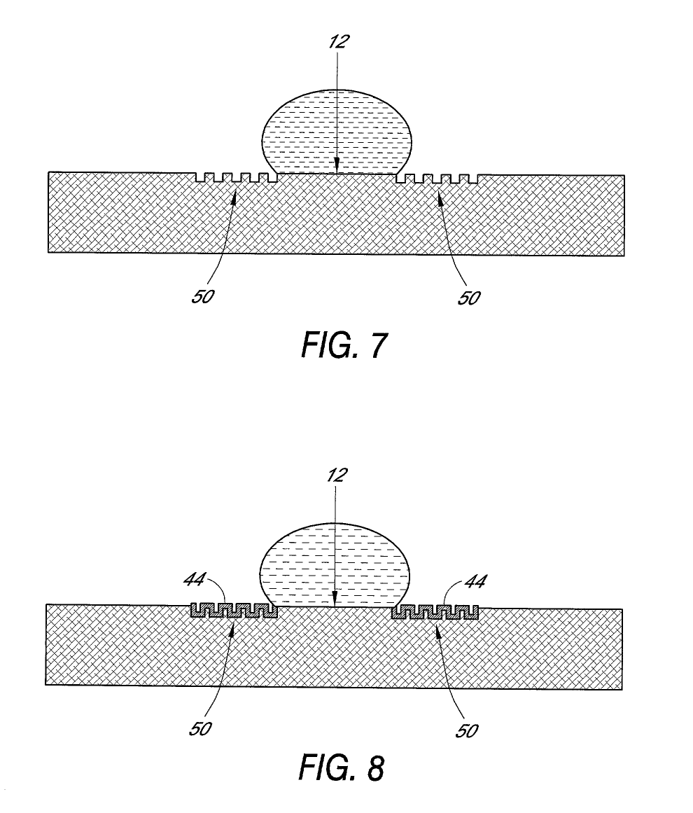

Finally, smooth and rough surfaces have different wetting properties. Surface control can be achieved by selectively patterning microroughness on the chip. In particular, a microroughened ring structure on the substrate can serve the same purpose as the hydrophobic Teflon ring as shown in FIG. 7. This Figure depicts an aqueous droplet positioned on the assay region 12. The droplet is held in place because the relatively smooth surface of the assay region 12 is more hydrophilic than the relatively rough surface of the microroughened ring 50 even though the surface material is the same. Preferably, the microroughness is accomplished by patterning and etching grooves on the surface using standard techniques in the art. The grooves can be square, rounded, angular, or of some other shape or combination of shapes. Preferably, the grooves are substantially uniform throughout the microroughened surface 50 and the size of the grooves is in the range of 10 Å to 10 μm in both width and depth.

Alternatively, microroughening can be used in conjunction with a hydrophobic material. FIG. 8 also shows a droplet being held in position on the assay region 12. Here, the area surrounding the assay region 12 is particularly hydrophobic as it is both a hydrophobic Teflon ring 44 and a microroughened ring 50. Preferably, the hydrophobic material (such as CYTOP or Teflon) is deposited on the surface first, and the microroughening is then performed directly on the hydrophobic material. The microroughening can be performed using a normal photolithography process and oxygen plasma to etch the grooves in the hydrophobic layer. As above, the grooves can be square, rounded, angular, or of some other shape or combination of shapes. Preferably, the grooves are substantially uniform and their size is in the range of 10 Å to 10 μm in both width and depth.

What is claimed is:

1. A method for positioning a plurality of droplets on electrodes, comprising:
providing a substrate having a plurality of electrodes onto which droplets can be positioned in a plurality of hydrophilic zones, wherein each hydrophilic zone is bounded by a hydrophobic zone; and
applying discrete aqueous droplets into a plurality of said hydrophilic zones.

2. The method of claim 1, wherein said hydrophobic zone comprises a fluoropolymer.

3. The method of claim 1, wherein said hydrophobic zone is continuous and completely encircles the hydrophilic zone.

4. The method of claim 1, wherein said hydrophobic zone comprises a line of hydrophobic material surrounding the hydrophilic zone, such that the hydrophilic surface of the substrate is exposed both inside of and outside of the hydrophobic line.

5. The method of claim 4, wherein the hydrophobic line is continuous.

6. The method of claim 4, wherein the hydrophobic line is broken.

7. The method of claim 1, further comprising forming the hydrophobic zone by applying a continuous layer of hydrophobic material on the substrate and then removing a portion of the hydrophobic material to expose the substrate.

8. The method of claim 7, wherein the hydrophobic material is removed by etching.

9. The method of claim 1, wherein the droplets contain reagents, and the applying step comprises applying different reagents to different zones on the substrate.

10. The method of claim 9, wherein the reagents are biological reagents.

11. The method of claim 10, wherein the reagents comprise DNA.

12. The method of claim 9, further comprising drying the reagents on the substrate to provide different dried reagents in different hydrophilic zones.

13. An assay surface, comprising:
a plurality of spatially discrete reagent zones, each comprising at least one reagent, wherein the reagent zones are relatively hydrophilic; and
a relatively hydrophobic line surrounding each of the reagent zones.

14. The assay surface of claim 13, further comprising relatively hydrophilic regions located outside of the hydrophobic lines, which regions do not comprise reagent.

15. The assay surface of claim 13, wherein the reagent in the reagent zones comprises DNA.

16. The assay surface of claim 13, comprising a plurality of different reagents respectively located in different of said reagent zones.

17. The assay surface of claim 13, wherein said substrate comprises a silicon wafer.

18. The assay surface of claim 13, further comprising a plurality of electrical conductors in functional contact with said reagent zones.

19. The assay surface of claim 18, wherein each of said reagent zones is respectively in contact with a different of said conductors.

20. The assay surface of claim 19, further comprising a continuous liquid layer overlying a plurality of said reagent zones.

21. The assay surface of claim 20, further comprising an electrode in electrical contact with said liquid layer, which layer is in electrical contact with a plurality of said reagent zones.

22. A method for performing an assay, comprising:
providing an assay surface comprising a plurality of reagent zones, each reagent zone surrounded by a hydrophobic material, wherein reagent is bound to the assay surface at the reagent zone, and hydrophilic areas are located on said surface both inside of and outside of the hydrophobic material;
flooding the assay surface with a liquid sample, such that a layer of liquid covers the assay surface; and
detecting an interaction between an analyte, if present, and the reagent in a reagent zone.

23. The method of claim 22, wherein the interaction of the reagent and the analyte produces an electrical signal measurable in said reagent zone.

24. The method of claim 23, further comprising measuring the electrical signal through one or more of a plurality of first electrodes in electrical contact with said reagent zones and one or more second electrodes in electrical contact with the liquid sample.

25. The method of claim 24, wherein the second electrodes are located remotely from the reagent zone in which said electrical signal is produced.

26. An assay device, comprising:
a substrate having a surface including plurality of reagent-bearing zones thereon, wherein the reagent-bearing zones are relatively hydrophilic and are each bounded by a relatively less hydrophilic zone, wherein the hydrophilic zones are differentiated from the less hydrophilic zones as a result of the texture of the surface in said zones.

27. The assay device of claim 26, in which the hydrophilic zone is smoother than the less hydrophilic zone.

28. The assay device of claim 26 in which the less hydrophilic zone comprises a fluoropolymer.

29. The assay device of claim 27 in which the less hydrophilic zone comprises a fluoropolymer.
